(19) United States
(12) Patent Application Publication
(10) Pub. No.: US 2008/0132723 A1
(43) Pub. Date: Jun. 5, 2008

(54) Process for the production of ethylene, acetic acid and carbon monoxide from ethane

(75) Inventors: Victor J. Johnston, Houston, TX (US); Joseph G. Duff, League City, TX (US); Debra Ann Ryan, League City, TX (US); Deborah R. Repman, Houston, TX (US); Melchior A. Meilchen, Houston, TX (US); David W. Townsend, Friendswood, TX (US); Cathy L. Tway, League City, TX (US)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(73) Assignee: Celanese International Corporation

(21) Appl. No.: 11/633,194

(22) Filed: Dec. 4, 2006

(51) Int. Cl. C07C 67/00; C09K 3/00

(52) U.S. Cl. 560/241; 252/372

(57) ABSTRACT

The present invention provides an integrated, optimized process for the production of ethylene, carbon monoxide and acetic acid from an ethane feed, including reacting ethane with oxygen in the presence of a catalyst to produce ethylene, carbon monoxide and acetic acid in a reactor, wherein the catalyst comprises the elements Mo, Pd, X, and Y in the gram-atomic ratios MOₐ Pd_{b} X_{c} Y_{d}, and the oxygen partial pressure in the reactor is about 15 psia to about 50 psia. The partial pressure of ethane in the reactor is about 50 psia to about 150 psia.

### FIELD OF THE INVENTION

The invention relates to the production of acetic acid, ethylene, and carbon monoxide. In particular, a method of selectively oxidizing ethane to acetic acid, ethylene, and carbon monoxide using a mixed oxide catalyst containing vanadium and tungsten or molybdenum is disclosed.

### BACKGROUND OF THE INVENTION

The oxidative dehydrogenation of ethane to ethylene in the gas phase at temperatures above 500° C. has been discussed, for example, in U.S. Pat. Nos. 4,250,346, 4,524,236, and 4,568,790. These patents are principally concerned with the preparation of ethylene. The use of mixed metal oxide catalysts to convert ethane to acetic acid is also described in patents and patent applications. For example, U.S. Pat. No. 5,162,578 describes a process for the selective preparation of acetic acid from ethane, ethylene, or mixtures thereof with oxygen in the presence of a catalyst mixture which comprises at least: (A) a calcined catalyst of the formula MoₓV_{y} or MoₓV_{y}Z_{Y}, in which Z can be one or more of the metals Li, Na, Be, Mg, Ca, Sr, Ba, Zn, Cd, Hg, Sc, Y, La, Ce, Al, Tl, Ti, Zr, Hf, Pb, Nb, Ta, As, Sb, Bi, Cr, W, U, Te, Fe, Co and Ni, and x is from 0.5 to 0.9, y is from 0.1 to 0.4, and z is from 0.001 to 1, and (B) an ethylene hydration catalyst and/or ethylene oxidation catalyst. The second catalyst component B is a molecular sieve catalyst or a palladium-containing oxidation catalyst.

An additional process for the preparation of a product comprising ethylene and/or acetic acid is described in European Patent No. EP 0 407 091 B1. According to this process, ethane and/or ethylene and a gas containing molecular oxygen is brought into contact at elevated temperature with a mixed metal oxide catalyst composition of the general formula AₐX_{b}Y_{c} in which A is Mo_{d}ReₑW_{f}; X is Cr, Mn, Nb, Ta, Ti, V and/or W; Y is Bi, Ce, Co, Cu, Fe, K, Mg, Ni, P, Pb, Sb, Si, Sn, Tl and/or U; a is 1; b and c are independently 0 to 2; d+e+f=a, and e is nonzero.

According to another process, ethane is oxidized to form ethylene (and some acetic acid) under relatively mild conditions at high selectivity and space-time yield using a catalyst having the formula MoₐVᵥTaₓTe_{y}. Preferably a is 1.0; v is about 0.01 to about 1.0; x is about 0.01 to about 1.0; and y is about 0.01 to about 1.0.

The selective preparation of acetic acid by catalytic gas-phase oxidation of ethane and/or ethylene in the presence of a palladium-containing catalyst is also described in commonly owned U.S. Pat. No. 6,399,816, which is incorporated by reference herein in its entirety. A gaseous feed comprising ethane, ethylene, and oxygen are brought into contact with a catalyst comprising the elements Mo, Pd, X, and Y in the gram-atomic ratios a:b:c:d in combination with oxygen: Moₐ Pd_{b} X_{c} Y_{d}. X represents one or more of Cr, Mn, Ta, Ti, V, Te, and W. Y represents one or more of B, Al, Ga, In, Pt, Zn, Cd, Bi, Ce, Co, Rh, Ir, Cu, Ag, Au, Fe, Ru, Os, K, Rb, Cs, Mg, Ca, Sr, Ba, Nb, Zr, Hf, Ni, P, Pb, Sb, Si, Sn, Ti and U. The indices a, b, c, and d represent the gram-atomic ratios of the corresponding elements: a=1, b=0.0001 to 0.01, c=0.4 to 1 and d=0.005 to 1.

Vinyl acetate is generally prepared commercially by contacting acetic acid and ethylene with molecular oxygen in the presence of a catalyst active for the production of vinyl acetate. Suitably, the catalyst may comprise palladium, an alkali metal acetate promoter and an optional co-promoter (for example, gold or cadmium) on a catalyst support. Integrated processes for producing vinyl acetate are provided by commonly owned U.S. Pat. No. 6,852,877, which is incorporated by reference herein in its entirety. U.S. Pat. No. 6,852,877 discloses a process for the production of vinyl acetate including (1) reacting ethane with oxygen in the presence of a catalyst to produce acetic acid (ethane oxidation), (2) reacting ethane with oxygen in the presence of a catalyst to produce ethylene (ethane oxidative dehydrogenation); (3) reacting the ethylene and acetic acid produced above with oxygen in the presence of a catalyst to produce a vinyl acetate product stream; and (4) separating the vinyl acetate from the product stream from step (3).

Also, commonly owned U.S. Pat. No. 6,790,983, which is incorporated by reference herein in its entirety, discloses a process for the production of vinyl acetate comprising (1) reacting ethane with oxygen in the presence of a catalyst to produce acetic acid and ethylene (ethane oxidation), (2) reacting the ethylene and acetic acid produced above with oxygen in the presence of a catalyst to produce a vinyl acetate product stream; and (4) separating the vinyl acetate from the product stream from step (2).

As described in these integrated vinyl acetate process references, using ethane instead of ethylene as a feedstock has significant cost advantages because ethane is available in natural gas. However, the ethane oxidation processes disclosed have the undesirable effect of producing some carbon oxides as a result of the over oxidation of ethane or oxidation of some of the product ethylene and/or acetic acid. Carbon monoxide so produced is particularly troublesome as it is a known poison of catalysts useful in the production of vinyl acetate from ethylene and acetic acid. In addition, the carbon oxides produced in the ethane oxidation step represent a loss of carbon efficiency that increases the waste gas generated by the process, the size and cost of the necessary equipment, and the amount of ethane feed required to make a specific amount of product.

Several attempts have been made to overcome this inherent limitation of the ethane oxidation processes described above. In U.S. Pat. No. 6,030,920, an attempt is made at minimizing the formation of carbon monoxide in the ethane oxidation step. However, this is accomplished by shifting the selectivity from carbon monoxide to carbon dioxide, such that while the amount of carbon monoxide produced is lowered, the total carbon lost to carbon oxides remains the same or increases. Thus, the problem of maximizing carbon efficiency remains.

Similarly, U.S. Pat. No. 6,852,877 provides a separate catalytic oxidation process wherein the carbon monoxide is converted to carbon dioxide and removed from the product gases prior to the vinyl acetate production step. This process overcomes the problem of carbon monoxide in the subsequent production of vinyl acetate, but again the overall carbon efficiency is not improved.

Additionally, both of the above methods result in increased production of carbon dioxide which results in increased green house gas emissions or requires additional processing equipment to capture the carbon dioxide.

An optimized ethane-based integrated vinyl acetate process that uses the ethane-containing mixture as a carbon feedstock is needed. Combining infrastructure, utilities, and other features such as a single feed gas compressor and off-gas scrubbing system is desirable compared to separate acetic acid and vinyl acetate processes that require individual feed gas compressors and off-gas scrubbing systems. Furthermore, an integrated process that reduces intermediate storage requirements is needed. In general, an optimized system to provide an integrated process for co-producing sales grade acetic acid and vinyl acetate in a flexible, integrated process based on ethane feedstock is desired.

### SUMMARY OF THE INVENTION

The present invention overcomes the problems described above by use of a catalyst and process conditions designed to optimize the production of acetic acid, ethylene, and carbon monoxide by the selective oxidation of ethane, separating the acetic acid from the product stream, further separating the carbon monoxide from the remaining product stream, recombining the acetic acid and ethylene containing streams in the presence of a suitable catalyst for the production of vinyl acetate. The carbon monoxide may be further utilized to produce, for example, additional acetic acid by carbonylation of methanol, thereby increasing the overall carbon efficiency of ethane to useful products.

### BRIEF DESCRIPTION OF THE SEVERAL VIEWS OF THE DRAWING

FIG. 1 is a schematic view of an embodiment of an acetic acid and vinyl acetate manufacturing process.

### DETAILED DESCRIPTION OF THE INVENTION

The present invention provides an integrated process for the co-production of acetic acid, vinyl acetate, and carbon monoxide from an essentially ethane feed, comprising the steps:

(A) reacting ethane with oxygen in the presence of a catalyst to produce acetic acid, ethylene and carbon monoxide;

(B) separating the acetic acid from the product stream;

(C) separating the carbon monoxide from the acetic acid-free product stream from step B, thus producing a carbon monoxide stream and an ethylene stream;

(D) reacting the ethylene from step C and the acetic acid from step B in the presence of a catalyst to produce vinyl acetate;

(E) utilizing the carbon monoxide stream from step C in another process or making it available for sale.

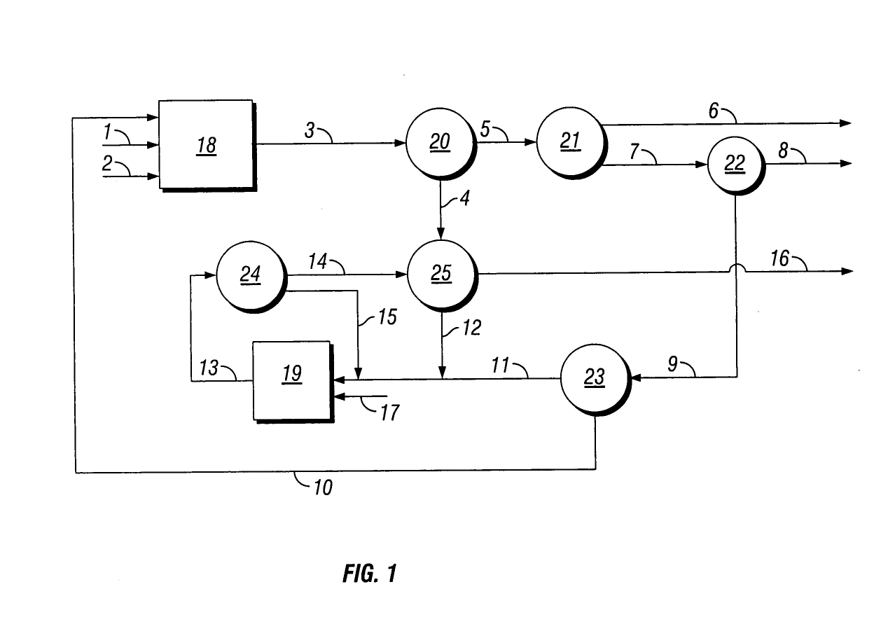

The process of the present invention will now be illustrated by reference to FIG. 1, which represents a simplified process flow diagram for a preferred embodiment of the present invention. Reactor 18 contains a catalyst active for the oxidation of ethane or mixtures of ethane and ethylene to ethylene, acetic acid, and carbon monoxide. A catalyst for reactor 18 has the elements Mo, Pd, X, and Y in the gram-atomic ratios MOₐ Pd_{b} X_{c} Y_{d}. X represents one or more of Cr, Mn, Nb, Ta, Ti, V, Te, and W. Y represents one or more of B, Al, Ga, In, Pt, Zn, Cd, Bi, Ce, Co, Rh, Ir, Cu, Ag, Au, Fe, Ru, Os, K, Rb, Cs, Mg, Ca, Sr, Ba, Nb, Zr, Hf, Ni, P, Pb, Sb, Si, Sn, Tl and U. The indices a, b, c, and d represent the gram-atomic ratios of the corresponding elements: a=1, b=0 to 0.01, c=0.4 to 1 and d=0.005 to 1.

A preferred catalyst for reactor 18 is Mo_{1.0} Pd_{0.00075} V_{0.55} Nb_{0.09} Sb_{0.01} Ca_{0.01}. The palladium concentration may be selected to be extremely low such as a subscript of about 0.0001 as disclosed in U.S. Pat. No. 6,399,816, filed on Oct. 9, 1998 and entitled, “Method for selectively producing acetic acid thought the catalytic oxidation of ethane,” which is incorporated by reference herein in its entirety. The catalyst may need to be used under reaction conditions for a short period of time to establish steady-state performance according to the invention. Such a break-in period is common in heterogeneous catalysts and does not typically impact the overall usefulness of the catalyst.

Reactor 18 is a fluidized bed or a fixed-bed reactor. The reactor is at a temperature of about 250° C. to about 400° C., preferably about 290° C. to about 350° C., and more preferably about 300° C. to about 320° C. The contact time within the reactor is about 4 to about 40 seconds, preferably about 4 to about 30 seconds. The gaseous feed comprises ethane or mixtures of ethylene and ethane which are fed to the reactor as pure gases or in admixture with one or more other gases. Examples of such additional gases are nitrogen, methane, carbon monoxide, carbon dioxide, air, and/or water vapor. The gas comprising molecular oxygen can be air or a gas comprising more or less molecular oxygen than air, e.g. purified oxygen.

The ratio of ethane/ethylene to oxygen is advantageously in the range from 1:1 to 10:1, preferably from 2:1 to 8:1. Relatively high oxygen concentration may be preferred. However, the partial pressure of the oxygen fed to reactor 18 may vary. For example, oxygen partial pressures of about 15 psia to about 50 psia may be selected. An oxygen partial pressure of about 20 to about 40 psia is preferred. To improve the selectivity to CO production, an amount of oxygen slightly lower than the maximum theoretically possible is desirable.

The external ethane feed 1 may be substantially pure (i.e. pipeline ethane) or slightly diluted with other gases like the ones generated by natural gas separation (e.g. 90 weight percent), or may be admixtures with one or more of nitrogen, carbon dioxide, hydrogen, and very low levels of C₃/C₄ alkenes/alkanes. Catalyst poisons like sulfur and acetylene should be excluded. The volume or percentage of inert components is only limited by economics. The partial pressure of ethane fed to reactor 18 may also vary. The partial pressure of ethane is about 50 psia to about 150 psia. An ethane partial pressure of about 80 psia to about 140 psia is preferred.

Oxygen feed 2 supplies oxygen to Reactor 1 and oxygen feed 17 supplies oxygen to reactor 19. Oxygen is preferably employed as pure oxygen but it may be employed as an oxygen containing gas mixture. Air or air enriched with oxygen may also be used. Water or water vapor may also be fed to reactor 18 either separately or as a mixture with one of the other feed streams (not shown in FIG. 1).

A recycle stream 10 comprising ethane or mixtures of ethane and ethylene is recovered from the process as described below and also fed to reactor 18. This recycle stream may be pure ethane or it may contain ethane in combination with other gases, e.g. nitrogen, carbon monoxide, carbon dioxide.

The effluent stream 3 of reactor 18 comprises primarily ethylene, acetic acid, carbon monoxide, carbon dioxide, water, and unreacted ethane. An aqueous acetic acid stream 4 is separated from the gas leaving reactor 18 by condensation and is further purified through conventional separation technologies such as a drying column and distillation processes in separator 25 to isolate an acetic acid product 12. Water is purged from the process in separator 25. The purified acetic acid is then available for sales or for use in a consuming process such as the vinyl acetate reactor 19 described below.

The remaining ethylene, carbon monoxide, carbon dioxide, and ethane stream 5 is then fed to separator 21, wherein carbon monoxide stream 6 is separated from the other gases that form stream 7, chiefly ethane, ethylene and carbon dioxide.

Carbon dioxide is removed from stream 7 in separator 22 to afford a carbon dioxide containing stream 8 and an ethylene and ethane containing stream 9. The sequential order of separations performed in separator 21 and 22 may optionally be reversed.

The ethylene and ethane containing stream 9 is directed to an ethylene/ethane separation unit, separator 23. This unit may be a cryogenic distillation column, adsorption unit, or any other suitable process. It is not required that the separation be complete. Thus, stream 10 comprising ethane may also contain appreciable amounts of ethylene, and stream 11 comprising ethylene may contain appreciable amounts of ethane. Stream 11 is combined with oxygen stream 17, acetic acid stream 12, and ethylene recycle stream 15 and the mixture is added to reactor 19.

A product stream 13 comprising vinyl acetate, water, ethane, gaseous by-products and unreacted acetic acid and ethylene is withdrawn from reactor 19 and is typically fed to a column 24 where a gaseous stream 15 including ethylene, ethane, inert gases, carbon monoxide, and carbon dioxide is withdrawn overhead. This gas stream may be split (not shown), with a first portion recycled as feed to reactor 18, and optionally a second portion fed to separator 22. A liquid stream 14 comprising vinyl acetate, water, unreacted acetic acid and possibly other high boiling point products of the process are withdrawn from the base of the column. Vinyl acetate is isolated from this stream. For example, the liquid stream may be fed to a distillation column where vinyl acetate and water is removed as an azeotrope, with acetic acid and other high boiling products being removed as a bleed from the base of the distillation column. The water in the overhead stream from the distillation column can be separated from the vinyl acetate in a decanter and a vinyl acetate product stream removed from decanter is purified. Alternatively, as shown in FIG. 1, stream 14 may be combined with stream 4 and the acetic acid, vinyl acetate, and water separation carried out in common. The acetic acid and ethylene and ethane streams 11, 12, and 15 and oxygen stream 17 are fed to reactor 19.

The towers, scrubbers, and routing referred to in the preceding paragraphs will have associated with them various heat exchangers, pumps, and connectors and will have operating parameters that are determined by the particular mixture of gases involved. It is within the ability of one of ordinary skill in the art to determine the proper configurations and parameters, given the above disclosure.

### EXAMPLES

During experimental analysis, several process conditions were tested. A system using catalyst with no palladium was compared to a system with a catalyst containing palladium. For these tests, reaction temperatures and contact times were adjusted to maintain oxygen conversion above 90%.

CHART 1
Comparison of Pd containing catalyst to catalyst containing
no Pd. Temperature is in ° C. Cnv % is conversion percent.

| | | Ethane | Oxygen | Nitrogen | Contact | | |
|---|---|---|---|---|---|---|---|
| Catalyst | Catalyst | mol % | mol % | mol % | Time, | Ethane | Oxygen |
| Composition | Temperature | Feed In | Feed In | Feed In | seconds | Cnv % | Cnv % |
| Pd 1 | 305.6 | 71.39 | 10.73 | 17.88 | 28.74 | 12.32 | 98.41 |
| Pd 2 | 294.4 | 72.02 | 9.93 | 18.05 | 29.56 | 11.28 | 93.69 |
| No Pd 1 | 346 | 73.98 | 14.84 | 11.17 | 18.06 | 15.95 | 98.03 |
| No Pd 2 | 340.9 | 73.98 | 14.85 | 11.17 | 18.24 | 15.9 | 97.49 |

CHART 2
Comparison of Pd containing catalyst to catalyst containing no
Pd using the same parameters as Chart 1. STY is space time yield
in grams product/liter catalyst/hour. Selectivities are based on ethane.

| | | | | | Acetic | |
|---|---|---|---|---|---|---|
| | Acetic | | | | Acid | Ethylene |
| Catalyst | Acid | Ethylene | CO2 | CO | STY | STY |
| Composition | Selectivity % | Selectivity % | Selectivity % | Selectivity % | G/L/Hr | G/L/Hr |
| Pd 1 | 25.15 | 60.24 | 9.1 | 5.48 | 58.97 | 65.39 |
| Pd 2 | 25.78 | 61.1 | 8.45 | 4.67 | 55.55 | 60.93 |
| No Pd 1 | 19.72 | 62.8 | 8.75 | 8.73 | 96.68 | 142.46 |
| No Pd 2 | 19.87 | 62.75 | 8.67 | 8.71 | 97.03 | 141.85 |

The catalyst without palladium had higher carbon monoxide selectivity as a function of COx selectivity, acetic acid space time yield, and ethylene space time yield during fixed bed and fluid bed testing. The data from Chart 1 and Chart 2 were collected under steady state conditions in a laboratory fluidized bed reactor.

A Pd containing catalyst was tested under steady state conditions in a laboratory fixed bed reactor system. Results from these tests are summarized in Charts 3-6.

CHART 3
Comparison of oxygen and ethane partial pressure and
selectivity. Cnv % is conversion percent.

| | Control | | | Contact | | |
|---|---|---|---|---|---|---|
| | Temp | P(C2H6) | P(O2) | Time, | Ethane | Oxygen |
| | ° C. | PSI | PSI | seconds | Cnv % | Cnv % |
| A | 320 | 93.48 | 39.56 | 5.98 | 14.28% | 48.69% |
| B | 320 | 98.49 | 20.06 | 5.80 | 18.42% | 93.25% |
| C | 320 | 69.56 | 37.68 | 8.05 | 28.20% | 83.32% |
| D | 320 | 136.30 | 41.87 | 7.09 | 20.16% | 84.04% |

CHART 4
Continuation of Chart 3. Selectivity and space time yield
results for four experimental conditions.

| | | | | | | Acetic | |
|---|---|---|---|---|---|---|---|
| | Acetic | | | | | Acid | Ethylene |
| | Acid | Ethylene | CO2 | CO | COx | STY | STY |
| | Selectivity % | Selectivity % | Selectivity % | Selectivity % | Selectivity % | G/L/Hr | G/L/Hr |
| A | 43.86% | 37.29% | 13.19% | 5.66% | 18.85% | 315.63 | 124.18 |
| B | 23.37% | 64.33% | 6.22% | 6.08% | 12.30% | 232.70 | 296.42 |
| C | 37.77% | 34.81% | 18.87% | 8.56% | 27.42% | 276.87 | 118.10 |
| D | 32.93% | 49.63% | 10.53% | 6.91% | 17.44% | 467.55 | 326.20 |

Conditions A and B illustrate the effect of oxygen partial pressure on oxygen conversion. Under the test conditions, decreased oxygen concentration provides a process for increased selectivity to desirable products: acetic acid, ethylene, and CO and decreased selectivity to CO2.

Condition C and D illustrate the effect of ethane partial pressure on COx selectivity and space time yield. Under the test conditions, increased ethane partial pressure yields a process with increased selectivity to desirable products.

CHART 5
Comparison of several process parameters and conversion
percentages. Cnv % means conversion percent.

| | Control | | | Contact | | |
|---|---|---|---|---|---|---|
| | Temp | P(C2H6) | P(O2) | Time, | Ethane | Oxygen |
| | ° C. | PSI | PSI | seconds | Cnv % | Cnv % |
| 1 | 305 | 97.11 | 41.01 | 5.51 | 12.55% | 39.17% |
| 2 | 320 | 93.48 | 39.56 | 5.98 | 14.28% | 48.69% |
| 3 | 305 | 135.91 | 41.89 | 6.92 | 12.73% | 52.86% |
| 4 | 305 | 100.26 | 21.14 | 5.60 | 12.25% | 58.09% |
| 5 | 305 | 96.47 | 40.39 | 8.34 | 17.08% | 59.43% |
| 6 | 320 | 110.84 | 41.43 | 5.36 | 18.34% | 67.23% |
| 7 | 305 | 137.63 | 41.52 | 8.43 | 16.29% | 71.09% |
| 8 | 305 | 135.49 | 22.34 | 6.32 | 14.33% | 81.15% |
| 9 | 310 | 70.00 | 37.57 | 11.86 | 28.47% | 81.74% |
| 10 | 320 | 69.56 | 37.68 | 8.05 | 28.20% | 83.32% |
| 11 | 320 | 136.30 | 41.87 | 7.09 | 20.16% | 84.04% |
| 12 | 305 | 95.90 | 20.28 | 8.90 | 19.90% | 89.09% |
| 13 | 320 | 98.49 | 20.06 | 5.80 | 18.42% | 93.25% |

CHART 6
Continuation of Chart 5. Additional process results including
selectivity and space time yield.

| | | | | | | Acetic | |
|---|---|---|---|---|---|---|---|
| | Acetic | | | | | Acid | Ethylene |
| | Acid | Ethylene | CO2 | CO | COx | STY | STY |
| | Selectivity % | Selectivity % | Selectivity % | Selectivity % | Selectivity % | G/L/Hr | G/L/Hr |
| 1 | 36.17% | 46.31% | 10.93% | 6.59% | 17.52% | 255.62 | 151.47 |
| 2 | 43.86% | 37.29% | 13.19% | 5.66% | 18.85% | 315.63 | 124.18 |
| 3 | 37.64% | 46.82% | 9.96% | 5.58% | 15.54% | 347.99 | 200.33 |
| 4 | 23.94% | 65.35% | 5.52% | 5.19% | 10.71% | 171.67 | 216.91 |
| 5 | 40.99% | 38.67% | 13.87% | 6.46% | 20.33% | 264.53 | 115.50 |
| 6 | 34.36% | 45.45% | 12.56% | 7.63% | 20.19% | 380.93 | 233.16 |
| 7 | 38.37% | 44.95% | 10.87% | 5.81% | 16.68% | 330.45 | 179.13 |
| 8 | 22.35% | 69.05% | 4.32% | 4.27% | 8.59% | 223.34 | 319.31 |
| 9 | 37.52% | 37.40% | 17.21% | 7.87% | 25.08% | 194.92 | 89.94 |
| 10 | 37.77% | 34.81% | 18.87% | 8.56% | 27.42% | 276.87 | 118.10 |
| 11 | 32.93% | 49.63% | 10.53% | 6.91% | 17.44% | 467.55 | 326.20 |
| 12 | 21.64% | 68.93% | 4.58% | 4.86% | 9.43% | 178.77 | 263.44 |
| 13 | 23.37% | 64.33% | 6.22% | 6.08% | 12.30% | 232.70 | 296.42 |

Charts 5 and 6 illustrate how process conditions can be manipulated to increase the selectivity to desirable products to greater than 80%. For example, temperature was varied from 305° C. to 320° C. Ethane partial pressure was varied from 70 to 138 psi and oxygen partial pressure was varied from 20 to 42 psi.

Also, several process parameters were compared to determine which factor most greatly influenced ethane and oxygen conversion as a prediction tool for controlling the performance behavior of the catalyst using a laboratory fixed bed reactor system. Control temperature was varied from 302° C. to 322° C. Partial pressure of oxygen was varied from 14 psia to 58 psia and partial pressure of ethane was varied from 46 psia to 157 psia. Contact time was varied from 4 seconds to 12 seconds. Oxygen partial pressure had the highest influence on ethane and oxygen conversion and ethane partial pressure had the second highest influence. Selectivity to ethylene, acetic acid, and carbon monoxide at over 90 percent was experienced when the ethane conversion was at 15-30 percent and the oxygen conversion was above 80 percent. The highest space time yield of ethylene was 390 g/L/hr and of acetic acid was 281 g/L/hr. Mathematical modeling of the process yielded similar results.

### Advantages

There are several advantages to this process. A process that is selective to carbon monoxide production relative to carbon dioxide production is overall more effective at converting oxygen and ethane and has the added benefit of requiring less equipment than a process that requires separate, independent systems for producing acetic acid and vinyl acetate. Selecting an oxygen concentration below the theoretical maximum has the advantage of reducing the need to take special precautions to avoid flammability. A reduced contact time also helps with process stability.

Another advantage of the inventive process is that the shift of (CO+CO₂) selectivity towards more CO and less CO₂, away from the direction historically taught as advantageous, has the beneficial effect of reducing the heat generation in Reactor 1. Since the size, equipment costs, and operating costs of Reactor 1 are highly dependent on the heat generated by the reaction, the shift in selectivity affords an improved process.

Yet another advantage of the inventive process is the reduction in the amount of carbon dioxide produced in converting the ethane into vinyl acetate in the integrated process. This results in lowered emissions of greenhouse gases. Additionally, what CO₂ is produced is captured in a form that makes it readily available for use or for sequestering, thereby decreasing the environmental impact of the process.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions or methods or in the sequence of the steps of the methods described herein without departing from the concept and scope of the invention. More specifically, it will be apparent that certain agents which are chemically related may be substituted for the agents described herein while the same or similar results would be achieved. All such similar substitutes and modifications apparent to those skilled in the art are deemed to be within the scope and concept of the present invention.

1. A method for the production of ethylene, carbon monoxide, and acetic acid using an ethane feed, comprising:
reacting ethane with oxygen in the presence of a catalyst to produce ethylene, carbon monoxide, and acetic acid in a reactor, wherein
the catalyst comprises the elements Mo, Pd, X, and Y in the gram-atomic ratios MOₐPd_{b}X_{c}Y_{d}, and
an oxygen partial pressure of the oxygen reacted with ethane is about 15 psia to about 50 psia; and
separating the carbon monoxide from the ethylene and acetic acid.

2. The method of claim 1, wherein the partial pressure of ethane is about 50 psia to about 150 psia.

3. The method of claim 1, wherein the reactor is a fixed bed reactor.

4. The method of claim 1, wherein the reactor is a fluid bed reactor.

5. The method of claim 1, wherein the reactor has a temperature of about 250° C. to about 400° C.

6. The method of claim 1, wherein the contact time within the reactor is about 4 seconds to about 40 seconds.

7. The method of claim 1, wherein the catalyst element X is selected from the group consisting of Cr, Mn, Nb, Ta, Ti, V, Te, W, and combinations thereof.

8. The method of claim 1, wherein the catalyst element Y is selected from the group consisting of B, Al, Ga, In, Pt, Zn, Cd, Bi, Ce, Co, Rh, Ir, Cu, Ag, Au, Fe, Ru, Os, K, Rb, Cs, Mg, Ca, Sr, Ba, Nb, Zr, Hf, Ni, P, Pb, Sb, Si, Sn, Tl, U, and combinations thereof.

9. The method of claim 1, wherein a is 1, b is 0 to 0.01, c is 0.4 to 1, and d is 0.005 to 1.

10. An integrated process for the production of vinyl acetate from an ethane feed, comprising:
reacting ethane with oxygen in the presence of a catalyst to produce ethylene, carbon monoxide, and acetic acid in a reactor;
removing the carbon monoxide; and
reacting the ethylene and acetic acid in the presence of a catalyst to produce vinyl acetate.

11. The method of claim 10, wherein
the catalyst comprises the elements Mo, Pd, X, and Y in the gram-atomic ratios MOₐPd_{b}X_{c}Y_{d}.

12. The method of claim 10, wherein an oxygen partial pressure of the oxygen reacted with ethane is about 15 psia to about 50 psia.

13. The method of claim 10, wherein the partial pressure of ethane is about 50 psia to about 150 psia.

14. The method of claim 10, wherein a reactor is a fixed bed reactor.

15. The method of claim 10, wherein the reactor is a fluid bed reactor.

16. The method of claim 10, wherein the reactor has a temperature of about 250° C. to about 400 ° C.

17. The method of claim 10, wherein the contact time within the reactor is about 4 seconds to about 40 seconds.

18. The method of claim 10, wherein the catalyst element X is selected from the group consisting of Cr, Mn, Nb, Ta, Ti, V, Te, W, and combinations thereof.

19. The method of claim 10, wherein the catalyst element Y is selected from the group consisting of B, Al, Ga, In, Pt, Zn, Cd, Bi, Ce, Co, Rh, Ir, Cu, Ag, Au, Fe, Ru, Os, K, Rb, Cs, Mg, Ca, Sr, Ba, Nb, Zr, Hf, Ni, P, Pb, Sb, Si, Sn, Tl, U, and combinations thereof.

20. The method of claim 10, wherein a is 1, b is 0 to 0.01, c is 0.4 to 1, and d is 0.005 to 1.
